(19) United States
(12) Patent Application Publication
(10) Pub. No.: US 2003/0008287 A1
(43) Pub. Date: Jan. 9, 2003

(54) Pheonotypic correlation process

(76) Inventors: Sarah Black, El Granada, CA (US); Stuart Hameroff, Tucson, AZ (US); Michael Weiner, Webster, NY (US)

Correspondence Address: GREENWALD & BASCH, LLP, 349 WEST COMMERCIAL STREET, SUITE 2490, EAST ROCHESTER, NY, 14445, US

(21) Appl. No.: 09/901,372

(22) Filed: Jul. 9, 2001

(51) Int. Cl. C12Q 1/68; G06F 19/00; G01N 33/48; G01N 33/50; G06K 9/00

(52) U.S. Cl. 435/6; 382/128; 702/20

(57) ABSTRACT

A process for determining the status of a living organism. In this process, cellular material which contains a first microtubule, a second microtuble, and a multiplicity of microtubule associated proteins is imaged to determine both the positions of the microtubles and the positions of the microtubule associated proteins located between the microtubules. The pattern of microtubules associated protein attachment is therein correlated with historic phenotypic data to determine whether there is any abnormality in the sample specimen.

### FIELD OF THE INVENTION

A process for imaging microtubule associated proteins, for determining certain specified cellular properties of such proteins, and for correlating such properties with phenotypic observations.

### BACKGROUND OF THE INVENTION

Microtubules are long, cylindrical tubes that are composed of bundles of small filaments, called protofilaments; protofilaments are formed by end-to-end association of tubulin molecules. Microtubules have an inside diameter of about 15 nanaometers, and an outside diameter of about 25 nanometers; and they are important component of the cytoskeleton.

Microtubles have been extensively discussed in the patent literature, especially with reference to Alzheimer's disease. Reference may be had, e.g., to U.S. Pat. Nos. 6,177,472, 6,174,890, 6,110,912, 6,107,104, 6,071,694, 6,010,913, 5,972,626, 5,968,936, 5,958,919, 5,900,375, 5,882,881, 5,877,173, 5,871,945, 5,869,262, 5,861,257, 5,837,853, 5,830,910, 5,830,659, 5,811,447, 5,811,243, 5,798,380, 5,629,163, 5,492,812, and the like. The entire disclosure of each of these United States patents is hereby incorporated by reference into this specification.

Microtubule associated proteins are high molecular weight proteins (with a molecular weight of from 200,000 to 300,000) that are associated with and enhance the polymerization of microtubules. Microtubules and microtubule associated proteins are dynamic, cellular components, undergoing continuous synthesis and degradation. Furthermore, the interactions between microtubules and microtubule associated proteins are also dynamic and transient. The properties and analyses of these microtubule associated proteins are discussed, e.g., in U.S. Pat. Nos. 6,190,522, 6,177,472, 6,166,190, 6,159,746, 6,156,764, 6,130,048, 6,121,004, 6,107,050, 6,106,824, 6,099,857, 6,075,026, 6,071,287, 6,107,050, 6,106,824, 6,099,857, 6,075,026, 6,071,694, 6,056,725, 6,040,168, 6,020,143, 6,020,140, 6,010,913, 6,005,088, 5,998,148, 5,994,304, 5,989,829, 5,985,578, 5,985,577, 5,981,279, 5,976,816, 5,968,936, 5,958,970, 5,955,970, 5,955,444, 5,955,312, 5,952,223, 5,945,291, 5,919,679, 5,886,025, 5,871,945, 5,861,257, 5,849,988, 5,846,220, 5,843,779, 5,837,853, 5,831,058, 5,811,243, 5,807,693, 5,795,735, 5,766,626, 5,744,354, 5,733,734, 5,705,501, 5,693,804, 5,686,269, 5,658,909, 5,654,189, 5,629,163, 5,616,608, 5,602,299, 5,601,985, 5,583,153, 5,580,898, 5,569,786, 5,492,812, 5,443,962, 5,443,952, 5,430,062, 5,427,931, 5,409,953, 5,356,928, 5,352,804, 5,336,684, 5,270,165, 5,213,962, 4,983,527, and the like. The entire disclosure of each of these United States patent applications is hereby incorporated by reference into this specification.

Applicants have discovered that there is a correlation between certain in vivo properties of microtubules and microtubule associated proteins and the status of the living organism in which they are disposed.

It is an object of this invention to provide a process for imaging the microtubules and the microtubule associated protein structure within an organism, for determining certain in vivo properties of such microtubules and such proteins, and for correlating the data so obtained with historical phenotypic data.

### SUMMARY OF THE INVENTION

In accordance with this invention, there is provided a process for determining the status of a living organism comprising the steps of sampling cellular material which contains microtubules and microtuble associated proteins, imaging said microtubules and microtubule associated proteins, determining the positions of said microtubules, determining the positions of said microtubule associated proteins, correlating the positions of said microtubules with historic phenotypic data, and correlating the positions of said microtubule associated proteins with historic phenotypic data.

### BRIEF DESCRIPTION OF THE DRAWINGS

The invention will be described by reference to the following drawings, in which like numerals refer to like elements, and in which:

FIG. 1 is a flow diagram of one preferred process of the invention; and

FIG. 2 is a flow diagram of one preferred imaging process of the invention.

### DESCRIPTION OF THE PREFERRED EMBODIMENTS

Microtubule associated proteins (MAPS) interconnect microtubules in specific fashions within cells to determine cell architecture and function, the current state of cell differentiation. The specific patter of attachment of MAPS on microtubules is thus a major determinant of cell condition. With the process of this invention, such pattern yields valuable information about the current state of health or disease of any particular cell, tissue, organ, or organism.

With the process of this invention, MAP attachment patterns within a variety of healthy and diseased cells are preferably detected; and the patterns do detected are preferably correlated with the cell's particular state.

In one embodiment, the MAPS patterns in vitro (i.e., in a solution containing microtubules and MAPS) are detected and analyzed. It is known that such in vitro MAPS patterns demonstrate periodic, “super-crystal” helical patterns; and they follow specific winding patterns intrinsic to the microtubule lattice structure.

In another embodiment, the MAPS patterns in cells are detected and analyzed.

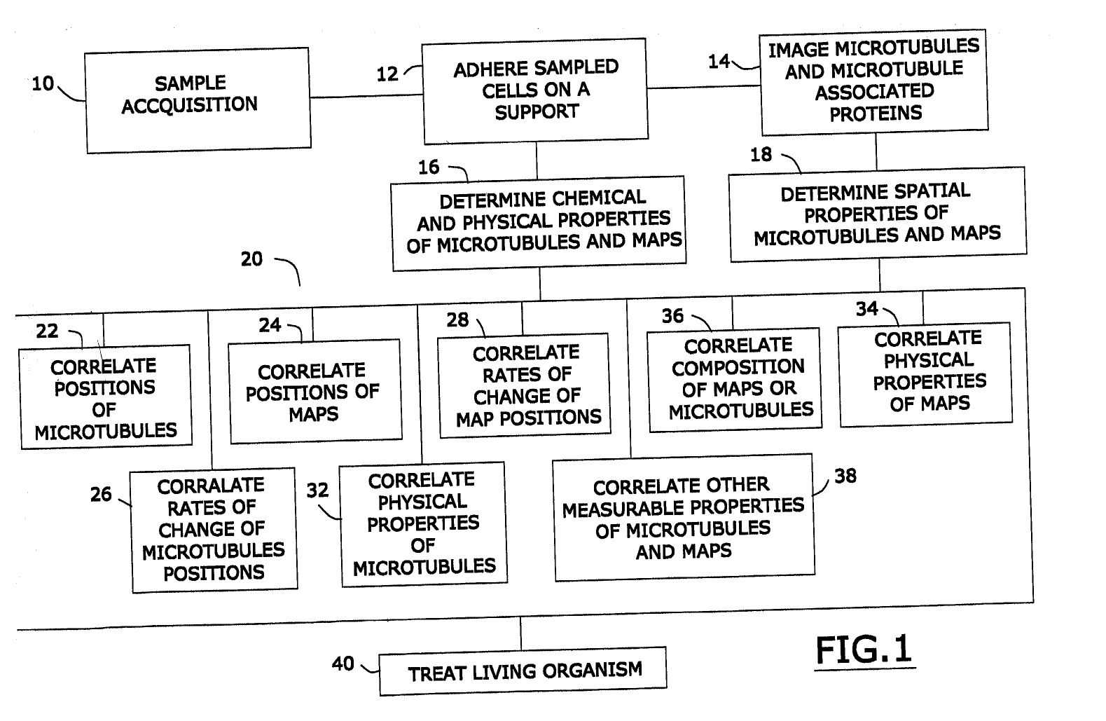

FIG. 1 is a flow diagram of one preferred process of the present invention. In step 10 of the process, a sample of cellular material is taken, preferably from a living organism. The sampling may be by conventional means, such as, taking blood.

Microtubules are disposed within all living, multicellular organisms. It is preferred, when conducting such sampling, to sample cells of the same differentiation state to produce comparative data. Thus, e.g., in one preferred embodiment, the microtubule associated protein pattern from a particular nerve cell is compared with the microtubule associated protein pattern of a comparable nerve cell.

In step 12 of the process, the cells that are sampled in step 10 are preferably adhered to a solid support, such as, e.g., a support consisting of polystyrene, or glass. As is known to those skilled in the art, cells have surface characteristics such that they adhere by means of electrostatic forces to inert substates.

The sampled cells which are adhered to an inert substrate are then prepared for measurement and analysis in step 12. This preparation and analyses may be conducted by conventional means. In one preferred embodiment, cells from any source are isolated and maintained as single cells.

In one embodiment, an image is produced of the pattern of the microtubule associated proteins (MAPS) of the microtubules of a specified cell sample.

The MAPS image may be produced by one of several distinct means, including, e.g., optical conductivity measurements, electrical conductivity measurements, X-ray crystallography, fluorescence labeling, immunofluorescence microscopy, dye or antibody staining, nuclear magnetic resonance, slow neutron imaging, quantum effect analyses, the use of nanoprobes, etc. Reference may be had, e.g., to U.S. Pat. Nos. 6,190,522, 6,177,472, 6,166,190, 6,159,746, 6,156,764, 6,136,992, 6,130,048, 6,121,004, 6,107,050, 6,106,824, 6,099,857, 6,075,026, 6,071,287, 6,107,050, 6,106,824, 6,099,857, 6,075,026, 6,071,694, 6,056,725, 6,040,168, 6,020,143, 6,020,140, 6,010,913, 6,005,088, 5,998,148, 5,994,304, 5,989,829, 5,985,578, 5,985,577, 5,981,279, 5,976,816, 5,968,936, 5,958,970, 5,955,970, 5,955,444, 5,955,312, 5,952,223, 5,945,291, 5,919,679, 5,886,025, 5,871,945, 5,861,257, 5,849,988, 5,846,220, 5,843,779, 5,837,853, 5,831,058, 5,811,243, 5,807,693, 5,795,735, 5,766,626, 5,744,354, 5,733,734, 5,705,501, 5,693,804, 5,686,269, 5,658,909, 5,654,189, 5,629,163, 5,616,608, 5,602,299, 5,601,985, 5,583,153, 5,580,898, 5,569,786, 5,492,812, 5,443,962, 5,443,952, 5,430,062, 5,427,931, 5,409,953, 5,356,928, 5,352,804, 5,336,684, 5,270,165, 5,213,962, 4,983,527, and the like. The entire disclosure of each of these United States patent applications is hereby incorporated by reference into this specification.

In one embodiment, electron miscroscopy is used to image the MAPS pattern. By way of illustration and not limitation, one may use the electron microscopy technique described in U.S. Pat. No. 6,177,472. The entire disclosure of this United States patent is hereby incorporated by reference in to this specification.

By way of further illustration, one may use the immunoelectron microscopy technique disclosed in U.S. Pat. No. 5,849,988;. The entire disclosure this United States patent is hereby incorporated by reference into this specification.

By way of yet further illustration, one may use slow neutron imaging to image the microtubles and the microtubule associated proteins.

Referring again to FIG. 1, in addition to imaging one or more positions of the microtubules and the microtubule associated proteins in step 16, the chemical and physical properties of the microtubules and the microtubule associated proteins may also be determined in step 16; and in step 18 the spatial properties of the microtubules and the microtubule associated proteins are determined. These analyses are conducted by conventional means such as, e.g., the protocols discussed in the patents discussed elsewhere in this specification.

As will be apparent to those skilled in the art, many types of data may be gathered regarding the microtubules and/or the microtubule associated proteins. Thus, by way of illustration and not limitation, one may gather data regarding the positions of MAP attachment to microtubules (by X-ray crystallography), the rates of change of MAP attachment to microtubules, the relative composition of MAPS (by protein isolation and mass spectrometry), the Qbit pattern of the tubulin conformation state, the speeds of the microtubule-assisted protein transport of protein secondary messengers, the destinations of microtubule-assisted protein transport of protein secondary messengers, the electrical and physical properties (current, voltage, etc.) of electrons, photons, elemental particles, etc. within microtubules, etc. The data thus gathered may be analyzed by directed graph or conceptual graph pattern analysis and/or other pattern extraction methods. In some embodiments, images are first digitized.

Once the chemical, physical, spatial, and other properties of the microtubules are determined in steps 14, 16, and 18, the data produced by these determinations are then correlated in step 20. The collected data can be correlated with other data such as, e.g., the genome of the organism, the gene expression profile of the cell, the change in the gene expression profiles of the cell, the differentiation states of the cell, the snRNA expression profiles, the cellular function, the age of the organism, the specific memories of the organism, pathological data of the organism (such as, e.g., sleep disorder data, psychiatric data, memory function data, disease state, etc.), and the like.

The comparison of the data collected with historical data then can be interpreted. Thus, e.g., one may examine whether the microtubule and MAP data collected from a test cell indicates differences from normal in one or more of the following ways: timing of gene expression through altered routing of secondary messengers, ability to differentiate, ability to communicate with other cells within the same organism, ability to interpret communications with other cells within the same organism, ability to communicate with other cells during embryogenesis or differentiation, ability to retain memory functions, ability to reconnect with other cells following injury or sleep, etc.

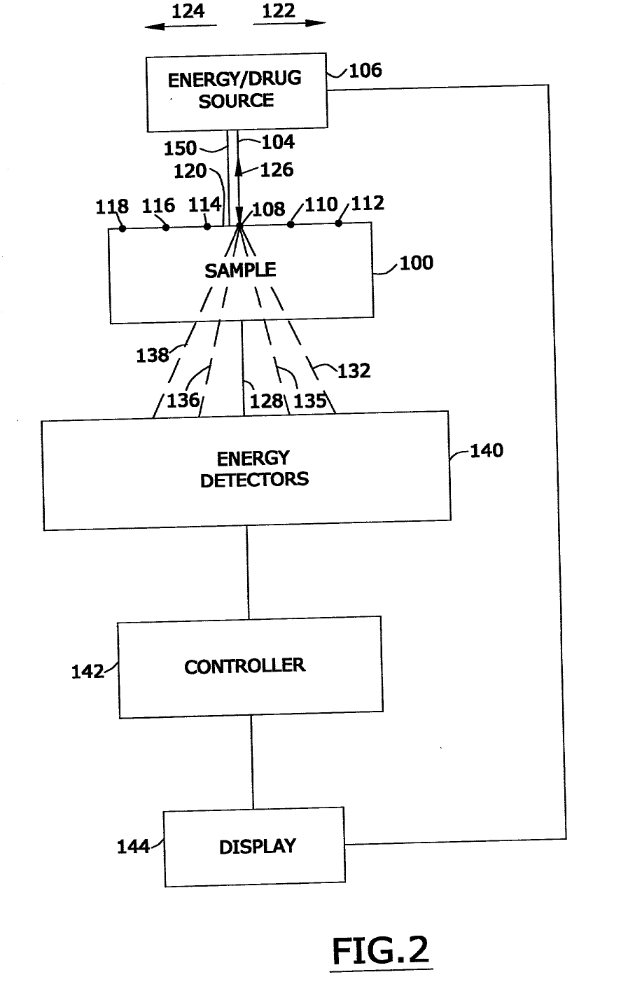

By way of illustration and not limitation, FIG. 2 is a schematic illustration of how the pattern of microtubule associated proteins (MAPS) on microtubules may be determined. Referring to FIG. 2, it will be seen that a microtubule sample (see step 12 of FIG. 1) is contacted with energy 104 from an energy source 106.

The energy source 106 is adapted to direct energy 104 to different points 108, 110, 112, 114, 116, 118, et al. on the surface 120 of the sample 100. In one embodiment, the energy source may be moved in the direction of arrow 122, arrow 124, and/or in a multiplicity of other directions in two or three planes so that, if desired, it can contact substantially every point on the surface 120 of the sample 100 with energy.

As will be apparent to those skilled in the art, different points 108, 110, 112, 114, 116, 118, etc. on surface 120 will have different configurations and/or compositions.

At some of the points 108 et al. a microtubule associated protein will be present. When a microtubule associated protein is available at one point, it may differ from a microtubule associated protein at another of such points either in its composition, its configuration, its density, or in one or more other properties.

At others of the points 108 et al., a microtubule associated protein will not be present. When a microtubule associated protein is not available at one point, such point may differ from another point at which a microtubule associated protein is not present, either in its composition, its configuration, its density, and/or one or more other properties.

Each point on the surface 120 of sample 100 is likely to have its own unique response to the energy 104. Thus, for example, at point 108 some of the energy 104 is reflected in the direction of arrow 126, some of the energy 104 is absorbed by the sample 100, energy 128 is transmitted, and energies 132, 134, 135, and 138 are diffracted.

The energy which is transmitted or diffracted through sample 100 will be detected by energy detector(s) 140 which may, e.g., be an array of photodectors. The information produced in detector(s) 140 is fed to controller 142 where it is digitally processed in the manner described below and, thereafter, displayed in display 144. Controller 142 is operatively connected to energy source 106; and, depending upon the response of the sample 100 to the energy 104, the location and/or intensity and/or frequency and/or the type of the energy source 104 may be varied.

When energy 104 is impacted at another point on the surface 120, another energy response pattern will be produced. Similarly, if the form or intensity of energy 104 is varied (such as, e.g., by substituting magnetic energy for light energy), the energy response pattern produced at a particular point will change. Thus, for any particular point on surface 120, a detailed energy response profile can be determined for any particular point by varying the frequency of the energy, the intensity of the energy, the type of the energy, etc.

The images produced by the process of FIG. 2, or by another imaging process, is preferably digitized so that they can be manipulated electronically. The production and analyses of digital images is well known. Reference may be had, e.g., to U.S. Pat. Nos. 6,207,878, 6,107,195, 6,207,190, 6,203,977, 6,203,546, 6,191,278, 6,191,120, 6,177,472, 6,177,121, 6,174,698, 6,150,089, 6,147,198, 6,136,335, 6,118,048, 6,110,673, 6,078,681, 6,060,238, 6,046,381, 6,046,007, 6,045,794, 6,030,442, 6,025,128, 6,020,189, 6,015,656, 6,007,994, 6,001,991, 5,994,529, 5,989,811, 5,982,534, 5,977,308, 5,968,960, 5,955,295, 5,949,899, 5,948,763, 5,939,281, 5,925,519, 5,902,732, 5,886,026, 5,874,059, 5,865,744, 5,830,645, 5,830,451, 5,827,660, 5,792,610, 5,784,162, 5,769,792, 5,764,819, 5,759,781, 5,750,335, 5,732,150, 5,720,928, 5,716,981, 5,698,549, 5,693,674, 5,691,384, 5,635,351, 5,610,027, 5,571,083, 5,532,139, 5,507,287, 5,503,986, 5,476,928, 5,449,767, 5,407,804, 5,394,268, 5,394,199, 5,330,911, and the like. The disclosure of each of these United States patents is hereby incorporated by reference into this specification.

Thereafter, the digitized images are manipulated and analyzed. Electronic manipulation and analysis of digitized images is a well known art exemplified by technologies such as the imaging analysis of overhead reconnaissance photography developed by the National Security Agency (NSA), National Reconnaissance Office (NRO), and other agencies, and used thereafter in conjunction with the National Institute of Health (NIH) to analyze radiological mammograms and detect correlations of bit patterns representing early stage breast cancer, well before human operators could identify them. The technology includes algorithms which adjust for overlaps in images, aspect ratio, relative position, etc., so that multiple images can be analyzed as they were one. Reference may be had, e.g., to U.S. Pat. Nos. 6,208,735, 6,206,691, 6,201,240, 6,184,858, 6,174,698, 6,161,031, 6,147,198, 6,141,434, 6,101,265, 6,078,681, 6,061,587, 6,028,910, 6,025,128, 5,993,001, 5,974,194, 5,909,478, 5,852,672, 5,845,639, 5,842,194, 5,841,892, 5,830,065, 5,802,361, 5,792,610, 5,760,829, 5,759,861, 5,740,267, 5,720,928, 5,586,196, 5,528,339, 5,503,986, 5,465,718, 5,438,989, 5,394,268, 5,319,550, 5,282,255, and the like. The disclosure of each of these United States patents is hereby incorporated by reference into this specification.

By way of further illustration, the image from a first cell, with a complex bit pattern representative of the nodes and connections on the microtubules of a cell, can be converted into a conceptual graph representation so that it can be mathematically analyzed and correlated other images, and patterns analyzed into higher order pattern representations. Thus, the digital data may be compared with data produced from the analyses of historical samples by means of a controller to determine to what extent, if any, the data is consistent or inconsistent. By way of illustration, bit map patterns and their higher order representations from multiple cells can be compared to other cells, and similarities, differences, and patterns can be identified and correlated. In order to make such comparisons, one may use technology similar to that used in face recognition and fingerprint comparison. Reference may be had, e.g., to U.S. Pat. Nos. 6,208,997, 6,200,022, 6,185,337, 6,184,926, 6,173,068, 6,160,923, 6,134,339, 6,128,398, 6,038,337, 6,038,333, 6,035,074, 6,002,209, 5,991,429, 5,990,901, 5,987,154, 5,982,914, 5,974,521, 5,963,670, 5,920,477, 5,913,205, 5,911,139, 5,893,095, reissue Pat. No. 36,041, U.S. Pat. Nos. 5,850,470, 5,844,573, 5,842,194, 5,838,839, 5,825,924, 5,809,322, 5,805,745, 5,774,129, 5,764,790, 5,719,950, and the like. The entire disclosure of each of these United States patents is hereby incorporated by reference into this specification.

In one embodiment, each cell in a biological organism is identified in a database that stores a variety of phenotype information about the position, the cell type, the organ, and the organism from which the individual cell was taken. Specific correlations of diseased and non-diseased cells from biopsies or other means of extraction (blood, skin cells, etc.) are analyzed to produce to determine how the properties of the microtubule associated proteins (MAPS) on a particular cell sample compares with that of normal cells.

One may evaluate one or more of several of the MAPS properties. Thus, one may evaluate the pattern(s) of the MAPS on the microtubules, their chemical composition(s), their dimensions, their spatial configurations, their physical properties, the moieties to which they are bonded or proximal to, and the like.

In one embodiment, the pattern of the MAPS on the microtubules is evaluated for the sample cells, and this pattern is then correlated with historical pattern data. Such correlation is therefore diagnostic and enables one to determine whether an organism has cancer or polycystic kidney disease, both of which maladies evidence themselves by abnormal MAPS patterning.

By way of illustration and not limitation, an abnormal MAPS pattern (or other abnormal MAPS property) can be correlated with genetic and phenotypic data such as age, sex, height, weight, species, pathologies, personality or behavioral data of the organism as a whole and with phenotypic data at a cellular level, such as differentiation state, gene expression profile, viral infection, cell cycle state or condition of viability by dint of environmental damage. The listing is merely illustrative, and many other correlatable phenomena will suggest themselves to those skilled in the art.

By way of further illustration, assume that a cancer cell has a specified MAP pattern. The MAP pattern from the known diseased cell can be correlated with the MAP pattern from a known healthy cell, thus revealing a pattern diagnostic of that cancer type. When other cells from the same organism are sampled their MAP patterns can be measured and compared to the two controls to determine if they, too, are tumorous.

Some of the measurable properties of the microtubules and/or the microtubule associated proteins which can be correlated with phenotypic data are illustrated in FIG. 1. Thus, e.g. in step 22 of the process depicted, one may correlates the positions of the microtubules vis-à-vis each other and other structures. In step 24 of the process, one may correlate the positions of the MAPS vis-à-vis each other, vis-à-vis the microtubules, and/or vis-à-vis other structures. In step 26 of the process, one may correlate the rates of change of the microtubule positions relative to other cellular structures; similarly, in step 28 of the process, one may correlate the rates of change turnover of the MAPS. The physical properties of the microtubules may be correlated in step 32; the physical properties of the MAPS may be correlated in step 34. The compositions/chemical properties of the microtubules may be correlated in step 36. Other measurable properties of the microtubules and/or the MAPS can be correlated in step 38.

Referring again to FIG. 1, and in step 40 of the process, a cell or cells with an abnormal MAPS and/or microtubule property or properties is preferably treated. Modeling with physics indicates that MAPS will reattach themselves using coherent phonon energies across a variety of spectrum, delivered by a variety of means. The coherent phonon energy's vibrational pattern, frequency, power, and the duration thereof, may be established through standard means of trail and error.

One may use multivariate analysis to determine the progression, over time, of disease and healthy states of cells throughout the body; and this multivariate analyses can be used to map the early causative effects of diseases such as, e.g. cancer. Reference may be had, e.g., to U.S. Pat. Nos. 6,208,942, 6,208,883, 6,201,991, 6,201,606, 6,197,288, 6,190,857, 6,183,752, 6,181,957, 6,174,997, 6,174,554, 6,172,744, 6,172,743, 6,168,933, 6,163,799, 6,157,677, 6,157,041, 6,156,515, 6,154,708, 6,154,560, 6,152,876, 6,146,897, 6,128,519, 6,122,042, 6,117,644, 6,117,290, 6,115,673, 6,107,103, 6,104,833, 6,097,495, 6,096,553, 6,095,982, 6,094,592, 6,091,843, 6,090,559, 6,087,662, 6,084,676, 6,078,389, 6,077,948, 6,076,406, 6,074,568, and the like. The entire disclosure of each of these United States patents is hereby incorporated by reference into this specification.

Once an abnormal cell has been identified, and referring again to FIG. 2, energy/drug source 106 can be suitably positioned over the cell and, via line 150, can deliver energy and/or drugs necessary to treat such cell.

One may treat the cell or cells or organism by means known to those skilled in the art. Thus, by way of illustration and not limitation, one may use the techniques described in one more of the patents described below.

One may treat the cell or cells by the process described in U.S. Pat. No. 4,106,488, the entire disclosure of which is hereby incorporated by reference into this specification. This patent discloses “A treatment of cancer by the application of external electromagnetic energy capable of the generation of heat in intracellular particles to induce selective thermal death of cancer cells in living tissue. This process allows for the selective treatment of cancer cells in living tissue without damaging the normal cells. The process comprises introducing minute particles into the interior of the cells of living tissue. These particles being injected intravenously while suspended in an appropriate solution are of a size generally having a diameter of approximately 1 micron or less and are of a material with properties, such as ferromagnetic, paramagnetic, or diamagnetic, so as to be inductively heated when subjected to a high frequency alternating electro_magnetic field. Introducing the particles as described, the patient is thereafter subjected to an alternating electromagnetic field to inductively heat the particles sufficiently to raise the temperature of the cells by an increment of 8.0. degree._{—}9.5. degree. Centigrade thus killing the cancer cells without harming the normal cells. Further selectivity and increased affinity of the cancer cells for these particles may be achieved by incorporating specific radioisotopes or tumor specific antibodies bound to these particles. These particles introduced intracellularly as described may be used as a method of delivering a chemotherapeutic agent primarily to the interior of the cancer cells by having the chemotherapeutic agent encapsulated within said particles and released at the proper time by application of the high frequency alternating electromagnetic field or by solubilizing the said particles within the cells.”

One may help measure and determine the properties of the cell or cells by the process disclosed in U.S. Pat. No. 4,136,683, the entire disclosure of which is hereby incorporated by reference into this specification. This patent discloses that: “The process comprises introducing minute particles into the interior of the cells. These particles being injected intraveneously while suspended in an appropriate solution are of the size generally having a diameter of approximately 1 micron or less and are of a material with properties such as ferromagnetic, paramagnetic, or diamagnetic. Shortly after being absorbed intracellularly, these particles will assume the same temperature as the respective cells which they have entered. It is a well established principle that the magnetic characteristics of ferromagnetic, paramagnetic, and diamagnetic materials vary as a function of their temperature. By measuring the magnetic characteristics of these particles shortly after they have entered the cell and with proper calibration, an exact determination of the temperature of the particle and therefore of the cell, can be made.”

By way of further illustration, and as is disclosed in U.S. Pat. No. 4,266,533 (the entire disclosure of which is hereby incorporated by reference into this specification), one may use “ . . . direct current, alternating current, and pulsed signals of single and double polarity. Invasive treatments involving the use of implanted electrodes have been followed, as well as noninvasive techniques utilizing electrostatic and electromagnetic fields. Much of the prior work that has been done is described in Volume 238 of the Annals of The New York Academy of Sciences published Oct. 11, 1974 and entitled “Electrically Mediated Growth Mechanisms in Living Systems” (Editors A. R. Liboff and R. A. Rinaldi). See also “Augmentation of Bone Repair by Inductively Coupled Electromagnetic Fields” by C. Andrew L. Bassett, Robert J. Pawluk and Arthur A. Pilla published in Volume 184, pages 575_{—}577 of Science (May 3, 1974). Basically, it has been established that, by changing the electrical and/or electrochemical environment of a living cell and/or tissue, a modification, often a beneficial therapeutic effect, of the growth, repair and maintenance behavior of said tissue and/or cells can be achieved. This modification or effect is carried out by subjecting the desired area of tissues and/or cells to a specifically encoded electrical voltage and concomitant current, whereby the interactions of charged species at the cells' surfaces are modified . . . ”

By way of further illustration, and referring to U.S. Pat. No. 4,314,554 (the entire disclosure of which is hereby incorporated by reference into this specification), it is disclosed that “ . . . a bone fracture area is a region of abnormally high electrical negativity which appeared to be associated with healing, and he along with Bassett found in 1964 that the application of an artifical electric field from a battery accelerated the healing. Becker found in 1963 and 1977 that silver electrodes, when driven positively, produce a germicidal environment, and he also found that electrical anodal currents of up to about 40 microamperes d.c. are beneficial for a germicidal effect but that negative electric currents of as little as one microampere d.c. are adequate for healing.”

Thus, e.g., one may use the process disclosed in U.S. Pat. No. 4,359,453 (the entire disclosure of which is hereby incorporated by reference), which discloses a treatment of atherosclerosis by the application of external electromagnetic energy capable of the generation of heat and biophysical alterations in intracellular particles and particles within atherosclerotic plaques to induce resolution of the atherosclerotic plaques. It is stated in this patent that: ““It has been known that there are certain physical differences that exist between atherosclerotic lesions and a normal blood vessel. One primary physical difference that exists is that atherosclerotic plaques and certain extravascular related lesions (xanthomas, corneal arcus) arise because altered endothelial permeability allows certain macromolecular plasma proteins (which are normally confined to the circulation i.e. lipids) to permeate endothelium and interact with charged components of the connective tissue gel of the vessel wall.”

One may use the process disclosed in U.S. Pat. No. 4,412,540, the entire disclosure of which is hereby incorporated by reference into this specification. This patent discloses that “For over forty years, high radio frequency electromagnetic radiation has been in regular use in the therapeutic treatment of a number of medical conditions. Many pathological processes have been successfully treated, by the direct application to the area under consideration, of an induced electromagnetic field in the VHF band. The Diapulse Corporation (New York, U.S.A.) produces a pulsed VHF electromagnetic field generator suitable for such medical use.”

U.S. Pat. No. 5,451,420, the entire disclosure of which is hereby incorporated by reference into this specifiation, discloses a miniature implantable magnetically actuated valve. This valve can be used in conjunction with the energy/drug source 106 depicted in FIG. 2.

One may cause the cell or cells to grow, as is described in U.S. Pat. No. 4,520,826, the entire disclosure of which is hereby incorporated by reference into this specification. This patent discloses ““A method for growth promotion in animals consists in percutaneous application of an electromagnetic field having a frequency of from 25 to 150 MHz and a power of from 30 to 40 W, to the region of the epididymal lobules of the spermatic cords and the epididymides, said application of an electromagnetic field being carried until a subcutaneous induration appears in the aforesaid region, that is, within an exposure time of from 10 to 20 s.”

By way of further illustration, one may use the cell modification disclosed in U.S. Pat. No. 528,265, the entire disclosure of which is hereby incorporated by reference into this specification. This patent discloses ““1. A cell modification process which comprises the steps of: (a) providing an element of metal at a culture medium having cells of a mammal therein including transformable cells chosen from the group consisting of fibroblast cells and malignant cells; (b) causing the element to introduce ions of the metal to the medium, the ions being substantially free of anions; (c) contacting the cells with the ions; (d) maintaining said contact for a sufficient period of time and with a sufficient amount of the ions to change the transformable cells to unspecialized cells characterized by their relatively multi_potent state; said period of time being greater than the time normally necessary to inhibit or render bacteriostatic bacteria in a wound with respect to fibroblast cells, and being less than the time normally necessary to kill Erlich's ascites fluid tumor cells with respect to free_floating malignant cells; (e) permitting or inducing a desired number of unspecialized cells to redifferentiate into cells of the culture medium other than fibroblast cells or malignant cells.”

One may also use the process disclosed in U.S. Pat. No. 4,556,051, which claims an “ . . . apparatus and method for promoting healing of injured tissue, such as fractured bone, with interacting electric current and a magnetic flux field . . . .” The entire disclosure of this United States patent is hereby incorporated by reference into this specification.

One may use the process of U.S. Pat. No. 4,590,822, the entire disclosure of which is hereby incorporated by reference into this specification. This patent describes “ . . . a method of treatment of infectious disease organisms comprising introducing minute particles into the interior of infectious cells. These particles possess ferromagnetic, paramagnetic or diamagnetic properties. After being localized intracellularly, these particles are inductively heated by application of an alternating electromagnetic field. The inductive heating is continued for a period of time sufficient to bring about an intracellular temperature rise to a minimum necessary to kill the infectious organism.”

One may apply radio frequency electromagnetic fields to wounded tissue or cells (diathermy) in accordance with U.S. Pat. No. 4,611,599, the entire disclosure of which is hereby incorporated by reference into this specification.

One may use the cancer treatment process disclosed in U.S. Pat. No. 4,662,359, the entire disclosure of which is hereby incorporated by reference into this specification. This patent describes “ . . . a process for the treatment of cancer in at least one region of host organism containing cancer cells and normal cells without substantially damaging living normal cells comprising: providing to a host organism minute particles capable of being inductively heated and of size capable of being absorbed into cancer cells, determining, after a period of metabolic time, the magnetic susceptibility of the particles with said region, subjecting the organism to an alternating electromagnetic field to inductively heat said particles at that point in metabolic time when the maximum difference in magnetic susceptibility between the cancer cell and normal cells within said region occurs, and continuing the inductive heating of said particles to attain an increase in intracellular temperature to selectively kill the cancer cells.”

One may use ultrasonic sound waves to disintegrate calculi, as is disclosed in U.S. Pat. No. 4,682,600, the entire disclosure of which is hereby incorporated by reference into this specification. ““Disintegrating calculi, such as kidney stones, by means of ultrasonic shock waves is known, for example, from German OS No. 3122056. The existence of a boundary surface effect between body tissue and the stone structure is exploited. Reflection of the incident shock wave at the front or rear boundary surface of the calculus generates pressure or tensile forces which shortly result in the shattering of the calculus.”\

Furthermore, one may use one or more of the processes of U.S. Pat. No. 4,815,447 (cancer treatment with photon energy), U.S. Pat. No. 4,846,181 (electrical stimulation to promote healing of soft tissue wounds, or injuries), U.S. Pat. No. 4,950,221 (seqential treatment of cells with a constant magnetic field and an alternating electromagnetic field), U.S. Pat. No. 4,974,114 (electrotherapy), U.S. Pat. No. 4,983,159 (necrosis of neoplasms), U.S. Pat. No. 4,993,413 (treating osteoporosis with a time varying magnetic field), U.S. Pat. No. 4,994,016 (electrical pulses using 30 herz energy), U.S. Pat. No. 5,014,699 (use of a signal having a series of substantially symmetric voltage cycles of bursted pulses having narrow pulse widths on the order of 0.5 to 20 microseconds), U.S. Pat. No. 5,045,050 (cancer treatment with a controlled, fluctuating, directionally oriented magnetic field parallel to a predetermined axis projecting through a malignant neoplasm), U.S. Pat. No. 5,067,495 (process for treating pain an electrical signal having a monophasic wave form comprising a series of sequential wave burst cycles), U.S. Pat. No. 5,0978,674 (electrical stimulation), U.S. Pat. No. 5,087,438 (treating diseases of the optic tract with a rotating magnetic field), U.S. Pat. No. 5,092,835 (healing brain cells with magnetic and electrical energy), U.S. Pat. Nos. 5,108,359, 5,135,466 5,147,284 (electromagnetic field radiator), U.S. Pat. Nos. 5,183,456, 5,197,140 (treating prostrate tumors with thermoseeds and radiation), U.S. Pat. Nos. 5,211,622, 5,261,422 (herpes treatement with sound), U.S. Pat. Nos. 5,295,494, 5,318,561 (controlling ion transport across a cell membrane with an applied oscillating magnetic field), U.S. Pat. No. 5,814,094 (ionphoteric system for promoting tissue healing), and the like. The entire disclosure of each of these United States patents is hereby incorporated by reference into this specification.

Applicants have discovered that microtubules are influenced by magnetic forces. Thus, when they are to be treated in the process of FIGS. 1 and 2, one can influence repositioning of microtuble associated proteins nodes by inducing a change in the MAP pattern by vibrational and other means, including coherent phonon energy.

Whatever treatment protocol is used, it will take advantage of the fact that the microtubules and the MAPS can be altered. If the test cell remained viable during data collection, then it may be treated and returned to the organism from whence it came. Otherwise, cells similar to the test cell but still resident in the sampled organism can be altered. Thus, e.g., one may change the amino acid sequence of tubulin or individual MAPS by alteration of gene sequence using gene therapy. Thus, e.g., one may change the MAP attachment pattern by treatment with coherent phonon energy. Thus, e.g., one may change the Qbit pattern within the microtubules. Thus, e.g., one may change the electrical properties of the microtubules (voltage, current, resistance, e.g.).

The microtubules and/or the MAPS may, upon treatment, have several different effects. Thus, e.g., such treatment may reprogram cells from a somatic state into stem cells, reprogram cells from one differentiation state to another, restore memory, restore reasoning ability, change gene expression, remove a pathological functioning state of one or more cells, etc.

It is to be understood that the aforementioned description is illustrative only and that changes can be made in the apparatus, in the ingredients and their proportions, and in the sequence of combinations and process steps, as well as in other aspects of the invention discussed herein, without departing from the scope of the invention as defined in the following claims.

We claim:

1. A process for determining the status of a living organism, comprising the steps of:
(a) sampling cellular material which contains at least a first microtubule, a second microtuble, and a multiplicity of microtubule associated proteins disposed between said first microtubule and said second microtubule,
(b) imaging said first microtubule, said second microtubule, and said multiplicity of microtubule associated proteins disposed between said first microtubule and said second mircotubule,
(d) determining the positions of said first microtuble, said second microtuble, and said multiplicity of microtubule associated proteins disposed between said first microtubule and said second microtubule,
(e) determining the pattern of said microtubule associated proteins disposed between said first microtubule and said second microtubule,
(f) comparing the positions said first microtuble, said second microtuble, and said multiplicity of microtubule associated proteins disposed between said first microtubule and said second microtubule, with historic phenotypic data, and
(f) comparing the pattern of said microtubule associated proteins disposed between said first microtubule and said second microtubule with historic phenotypic data.

2. The process as recited in claim 1, wherein said positions of said first microtuble, said second microtuble, and said multiplicity of microtubule associated proteins disposed between said first microtubule is determined by X-ray crystallography.

3. The process as recited in claim 1, further comprising the step of determining the rates at which said multiplicity of microtubule associated proteins change said positions.

4. The process as recited in claim 1, further comprising the step of determining the composition of said microtubule associated proteins.

5. The process as recited in claim 4, wherein said compositions of said microtubule associated proteins are determined by protein isolation.

6. The process as recited in claim 4, wherein said compositons of said microtubule associated proteins are determined by mass spectrometery.

7. The process as recited in claim 1, further comprising the step of determining the Qbit pattern of the tubulin conformation state, wherein said first microtubule and said said second microtubule is each comprised of said tubulin.

8. The process as recited in claim 1, further comprising determining the speeds of microtubule-assisted protein transport of protein secondary messengers.

9. The process as recited in claim 1, further comprising determining the destinations of microtubule assisted protein transport of protein secondary messengers.

10. The process as recited in claim 1, further comprising adhering said sampled cells to a solid support.

11. The process as recited in claim 10, wherein said solid support is comprised of polystyrene.

12. The process as recited in claim 1, wherein said sampled cells are isolated and maintained as single cells.

13. The process as recited in claim 1, wherein said multiplicity of microtubule associated proteins are imaged by slow neutron imaging.

14. The process as recited in claim 1, further comprising the step of digitizing data produced by said imaging said first microtubule, said second microtubule, and said multiplicity of microtubule associated proteins disposed between said first microtubule, thereby producing digitized data.

15. The process as recited in claim 14, further comprising the step of electronically manipulating said digitized data.

15. The process as recited in claim 14, further comprising the step of analyzing said digitized data.

16. The process as recited in claim 1, further comprising the step of treating said living organism.

17. The process as recited in claim 16, wherein said living organism is treated by the application of external electromagnetic energy.

18. The process as recited in claim 16, wherein said living organism is treated with coherent phonon energy.

19. The process as recited in claim 16, further comprising the step of changing the attachment pattern of said microtubule associated proteins.

20. The process as recited in claim 16, further comprising the step of changing the amino acid sequence of indvidual microtubule associated proteins.
